(19) United States
(12) Patent Application Publication
(10) Pub. No.: US 2010/0174298 A1
(43) Pub. Date: Jul. 8, 2010

(54) Surgical Instrument to Ease Stitching

(76) Inventor: Maurizio Ceschin, Trieste (IT)

Correspondence Address: FLYNN THIEL BOUTELL & TANIS, P.C., 2026 RAMBLING ROAD, KALAMAZOO, MI, 49008-1631, US

(21) Appl. No.: 12/224,026

(22) PCT Filed: Feb. 19, 2007
(86) PCT No.: PCT/IT2007/000108

§ 371 Date: Nov. 3, 2008

(30) Foreign Application Priority Data
Feb. 20, 2006 (IT) TS2006U000003

(51) Int. Cl. A61B 17/04

(52) U.S. Cl. 606/148

(57) ABSTRACT

The instrument include a body (1) consisting of a steel blade, two arms (2,3) and a sleeve member (3.1) which glides on body (1); the first arm (2) is fixed on an end of the body (1); the second arm (3) is fixed to the sleeve member (3.1); both the arms (2,3) are perpendicular to the body (1) and have on their extremities a hook (2.1, 3.2) with a rounded point; the two hooks (2.1, 3.2) mirror each other and have opposite concavity; the sleeve member (3.1) has an auto-blocking system that inhibits autonomous sliding along body (1); both the hooks (2.1, 3.2), once under tension, grab the skin bringing together the lips of the wound for stitching in a precise and symmetric manner avoiding folds and overlap.

### FIELD OF APPLICATION

This invention relates to a surgical instrument and in particular a stretching device used for stitching the skin and the subcutaneous layers.

### STATE OF THE ART

A problem noted in the field of surgery is that of perfecting the stitching of the subcutaneous layers and the skin both in terms of avoiding infection as well as unaesthetic scars. It is therefore necessary that the lips of the wound are perfectly aligned before and during the stitching. Currently this alignment is obtained thanks to an assistant who, using two hands or two hooks (one per hand), stretches the skin by pulling at the extremities of the wound and causing the lips to come together. For the surgical assistant it is incommodious to maintain a stable alignment of the lips of the wound for the time necessary to complete the stitching. In addition, the use of an assistant is a waste of human resources. Obviously if the surgeon is alone in the phase of closing the wound, the stitching may be done in an approximate manner. To help the surgeon who works alone some surgical instruments have already been proposed. U.S. Pat. No. 4,753,237 by Puchy describes a surgical instrument that facilitates alignment of the lips of a wound for stitching. This instrument consists of a flexible bar with hooks on the extremities. The hooks grab the extremities of the wound or incision. Bending the elastic bar which tends to straighten itself, a tension is caused on the two hooks provoking a stretching of the skin and the desired alignment. However this instrument is produced with a single-use plastic and is therefore costly. It creates interference in the operating field as the arc of the bar reduces the maneuverability over it. Additionally the extremities of the bar superimpose the vertices of the wound, creating another obstacle for the surgeon. Exactly of this reason the instrument is rarely or never appropriate usable for small wounds. The tension on the hooks is poorly modulated and the instrument is difficult to use on irregular surfaces.

Also cited is the U.S. Pat. No. 4,344,420 by Forder that describes an instrument for kidney surgery that consists of a pair of arms mounted on a cross member. One of the arms is fixed to the cross member at one end, while the other arm has an opening in which slides the element itself and a locking mechanism. Each arm is fitted with a blade mean extending from its free end. Both blade means are placed on the median portion of the lips in such a way that the axis of opening is perpendicular to the major axis of the wound. Additionally the mobile arm has a quite complex locking mechanism consisting of aligning holes with pin-nosed pawl locking device. The locking mechanism requires maintenance and is difficult to clean.

### SUMMARY OF THE INVENTION

It is therefore an object of the present invention to provide a surgical instrument designed to ease stitching without the defects of the instruments now in existence.

This and other objects are solved by a surgical instrument as claimed in claim 1.

The surgical instrument consists of a body on which are mounted two arms.

The body consists of rectilinear steel blade generally quadrangular in cross section.

The first arm is fixed to an end of the body, preferably forming a perpendicular with the body. The second arm is fixed to a sleeve member slidably mounted on the body. The cross-section of the sleeve member is analogous to that of the body and so preferably quadrangular. The sleeve member has an auto-locking system. The end opposite to that, which the first arm is fixed is rounded thereby facilitating the introduction of the sleeve member on the body.

Each of the arm has a hook extending therefrom their free end not engaging the body. The longitudinal axis of each hook is perpendicular to the arm from which it originates. The concavity of the two hooks are opposite each other. The hook of the arm fixed to the body mirrors the hook on the arm which slides oil the body. The curvature of each hook is so designed so to take only the layers of skin and subcutis, anatomical structures that render the skin mechanically resistant. The point of the each hook is rounded thus avoiding trauma to the hypodermic tissue, in particular vessels and nerves.

Both the hooks once in traction' hook the extremities of the wound or incision causing the lips to come together in a precise and symmetric manner avoiding folds and overlap.

It is a simple instrument, easy to clean, autoclavable and does not require maintenance.

The use of the instrument is particularly useful when the skin is very elastic and/or abundant, with consequent uneven cutaneous layers and loss of physiological anatomic architecture. In addition its use is advantageous for body segments that include cutaneous folds (neck, armpit, cubital region, groin, buttocks, popliteal region and ankle).

The surgical instrument has in addition the following advantages:
speeds up the positioning of suture of any subcutis and cutis types: metal graph, thread, skin closures and glue;
simplifies the surgeon's work no longer having to take care to align the lips of the wound;
saves on human resources, rendering unnecessary an assistant to hold the skin in the regions difficult to stitch;
improves the aesthetic result of the stitching;
reduces post-surgery infection from delayed-healing.

### BRIEF DESCRIPTION OF DRAWINGS

Further characteristics and advantages of the instrument will appear from the following description taken together with the accompanying drawings:

FIG. 1 shows a lateral view of the instrument;

FIG. 2 shows a second lateral view of the instrument;

FIG. 3 shows an inferior view of the instrument;

FIG. 4 shows with great detail a frontal view of a part of the instrument;

FIG. 5 shows a cross section of the sleeve member;

FIG. 6 shows a perspective view of the instrument;

FIG. 7 shows a second perspective view of the instrument;

FIG. 8 shows a third perspective view of the instrument;

FIG. 9 shows a perspective view of the instrument in operation.

### DETAILED DESCRIPTION OF AN EXAMPLE OF PREFERRED EMBODIMENT

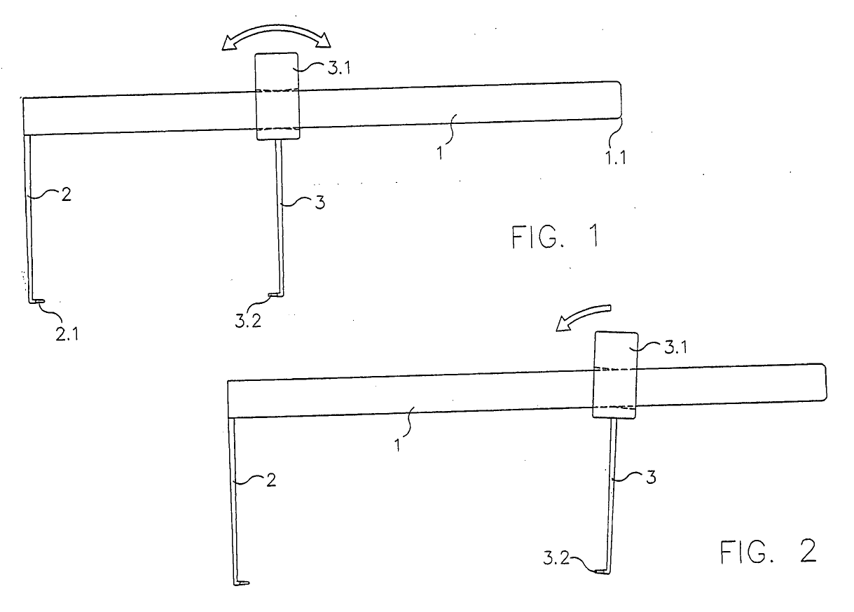

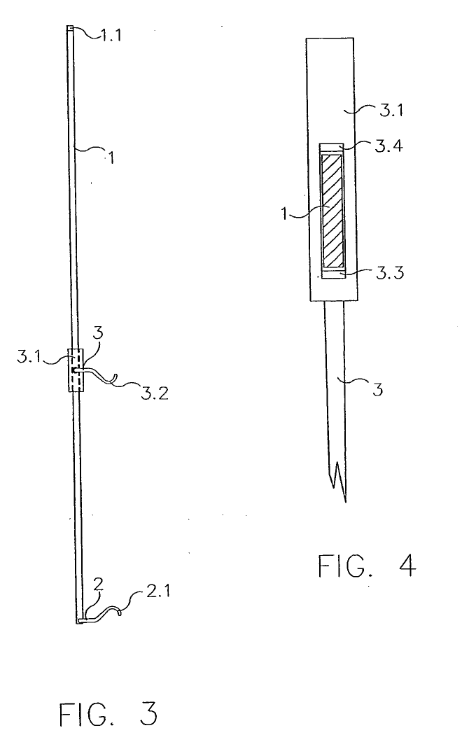

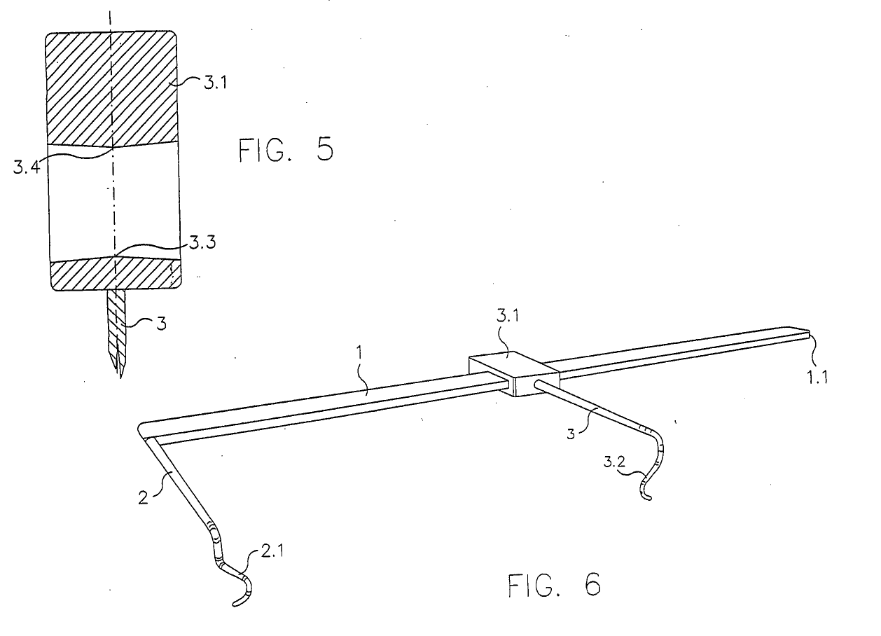

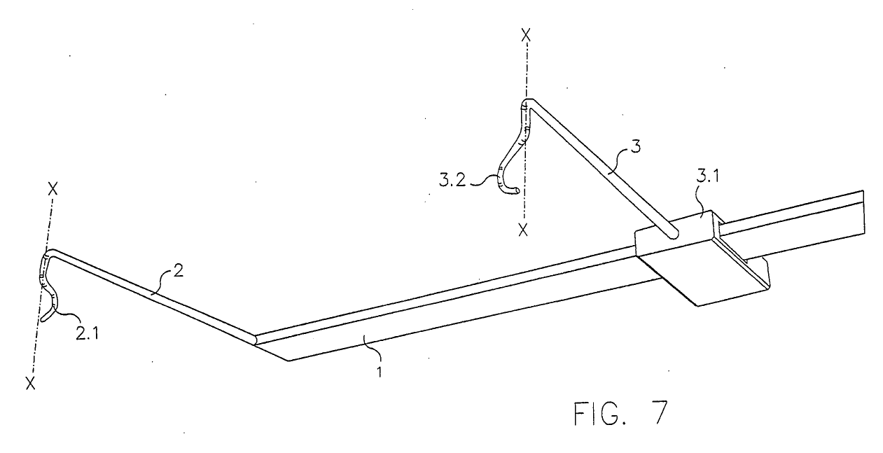

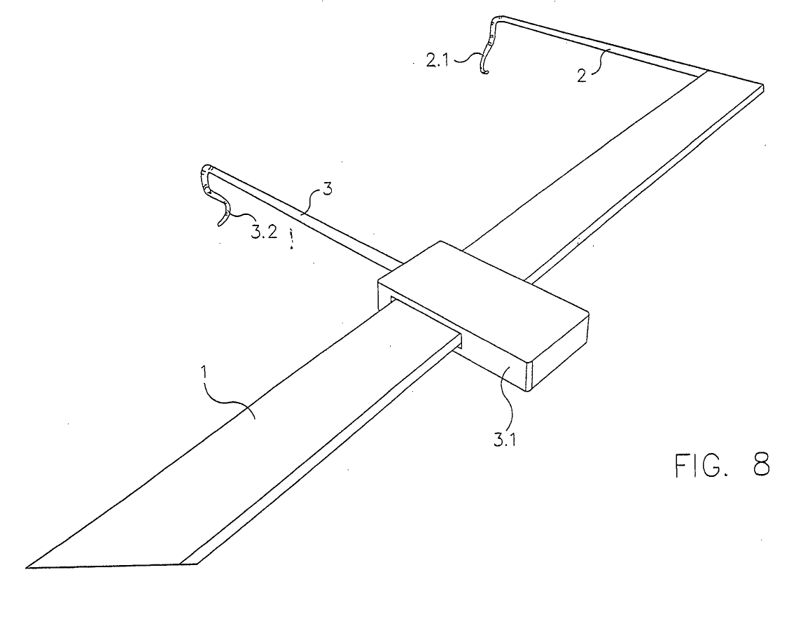

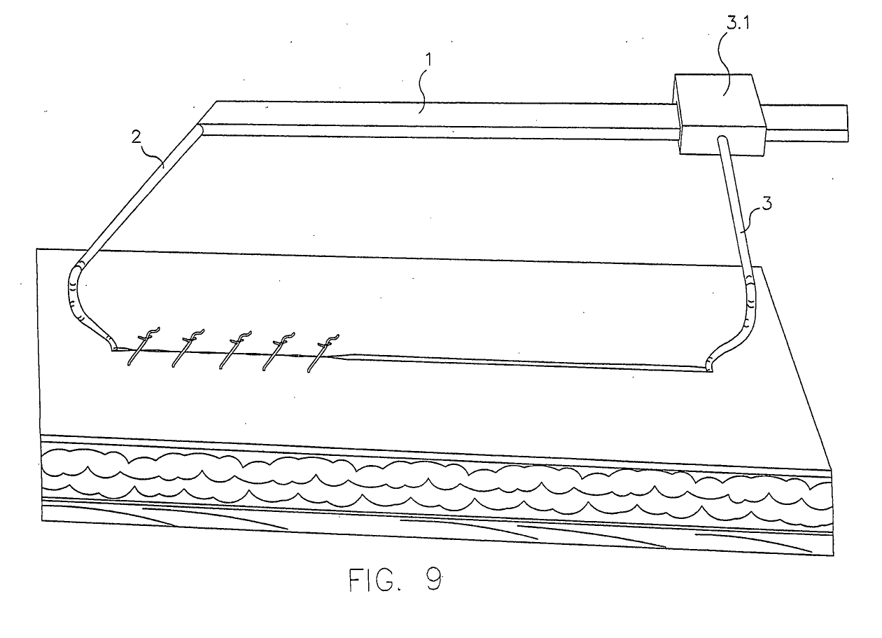

Referring to FIGS. 1-8, the instrument includes a body 1, two arms 2, 3 and a sleeve member 3.1.

The body 1 is a steel rectilinear blade with a rectangular cross-section, therefore having two minor lateral faces and two major lateral faces.

The first arm 2 is fixed to an end of the body 1 and has a hook 2.1 on the end not fixed to the body 1. The first arm 2 is perpendicular to body 1. The longitudinal axis x-x of the hook 2.1 is perpendicular to the first arm 2. The hook 2.1 has a rounded point. The arm 2 is fixed to a minor lateral face of the body 1. The second arm 3 is fixed to a sleeve member 3.1 with a rectangular cross-section that slides on body The introduction of the sleeve member 3.1 on the body 1 is facilitated by the presence of rounding 1.1 on the minor sides of the end of body 1 opposite that to which arm 2 is fixed. The arm 3 is fixed to the sleeve member 3.1 in a position such that its longitudinal axis is perpendicular to that of the body 1 and rests on the same minor lateral face of the body 1.

Also the arm 3 fixed to the sleeve member 3.1 has a hook 3.2 on the end not fixed to the sleeve member 3.1. The longitudinal axis x-x of the hook 3.2 is perpendicular to the second arm 3. The hook 3.2 has a rounded point. The two arms 2, 3 rest on the same minor lateral face of body 1 and, when the instrument is not in operation, they are perpendicular to body 1. The hook 2.1 of arm 2 fixed to body 1 mirrors-hook 3.2 of arm 3 fixed to the sleeve member 3.1. The two hooks 2.1, 3.2 have opposite and diverging concavity.

The internal opening of the sleeve member 3.1 is over-sized with respect to the body 1, allowing in this way the sleeve member 3.1 to rotate a few degrees with respect to the longitudinal axis of body 1. One of the minor faces of the internal opening of the sleeve member 3.1 that faces one of the minor faces of body 1 is filed away on the external portions to form a central cusped 3.3. Also the second minor faces of the internal opening of the sleeve member 3.1 is filed away on the external portions to form a cusped 3.4, that however is nearly central and shifted a few millimetres with respect to the other cusped 3.3.

The instrument is used positioning the hook 2.1 of the arm 2 fixed to body 1 in an end of the wound; then the sleeve member 3.1 is slid along the body 1 until the hook 3.2 of arm 3 fixed to the sleeve member is positioned at the opposite end of the wound. Modulating the pressure exerted manually to the arms 2, 3 to distance one from the other the desired level of tension on the lips of the wound is obtained. More precisely, exerting a light pressure the lips of the wound come together without causing much tension on the skin, permitting the positioning of the stitches in the subcutis. With further pressure on arms 2, 3 a marked tension along the lips of the wound is created guaranteeing a perfect alignment of the lips of the wound thus easing the stitching with scarce margins of error, also on the part of less expert surgeons.

The arm 3 fixed to the sleeve member 3.1 cannot move closer to the fixed arm 2 on its own. In fact, the force of the end of the wound on the hook 3.2 and consequently on arm 3 fixed to the sleeve member 3.1 causes a rotation of the sleeve member 3.1 and the assumption of a direction slightly inclined with respect to the longitudinal axis of body 1. The minor internal faces of the sleeve member 3.1, and more precisely the two non-symmetric cuspeds 3.3, 3.4 come in contact with the minor lateral faces of body 1. The friction provoked by the cuspeds 3.3, 3.4 induces the sleeve member 3.1 on body 1 to auto-block.

At the end of the stitching operation, the arm 3-sleeve member 3.1 unit are made to rotate to the unlocking position. Lacking the contact between the minor faces of body 1 and the minor internal faces of the sleeve member 3.1, the latter is permitted to slide freely and thus distancing the hook 3.2 of the arm 3 fixed to the sleeve member 3.1 from the closed wound. In the end the hook 2.1 is also removed and the instrument taken away from the operating area.

1. Surgical instrument to ease stitching including a body (1), two arms (2, 3) and a sleeve member (3.1); the body (1) consisting of a steel rectilinear blade with a rectangular cross-section; the first arm (2) is fixed to an end of the body (1), with its own longitudinal axis perpendicular to body (1); the first arm (2) having on the end not fixed to the body (1) a hook (2.1) of which the longitudinal axis is perpendicular to the first arm (2) and has a rounded point; the instrument is characterized by the fact that the second arm (3) is fixed to a sleeve member (3.1) sliding on body (1) having a rectangular cross-section; the arm (3) is fixed to the sleeve member (3.1) in a position such that its longitudinal axis is perpendicular to that of the body (1) when the instrument is not operating; the arm (3) fixed to the sleeve member (3.1) having on its end not fixed to the sleeve member (3.1) a hook (3.2) of which the longitudinal axis is perpendicular to the arm (3) fixed to the sleeve member (3.1); the hook (3.2) has a rounded point; the hook (2.1) of arm (2) fixed to body (1) mirrors hook (3.2) of arm (3) fixed to the sleeve member (3.1) and the two hooks (2.1, 3.2) have opposite and diverging concavity; the two arms (2, 3) insist to the same lateral face of body (1); the internal opening of the sleeve member (3.1) is oversized with respect to the cross-section of body (1), allowing in this way the sleeve member (3.1) to rotate a few degrees with respect to the longitudinal axis of body (1); one of the faces of the internal opening of the sleeve member (3.1) that faces one of the faces of body (1) is filed away on the external portions to form a central cusped (3.3); the face of the internal opening of the sleeve member (3.1) opposite to that presenting the central cusped (3.3) is also filed away on the external portions to form a cusped (3.4); this second cusped (3.4) is nearly central and shifted a few millimetres with respect to the other cusped (3.3); when the instrument is operating, the force of the end of the wound on the hook (3.2) of the arm (3) fixed to the sleeve member (3.1) causes a rotation of the sleeve member (3.1) and the assumption of a direction slightly inclined with respect to the longitudinal axis of body (1); the cuspeds (3.3, 3.4) of the sleeve member (3.1) come in contact with the lateral faces of body (1) and the friction induces an auto-block of the sleeve member (3.1) on body (1).

2. Surgical instrument, according to claim 1, characterized by the fact that the end of the body (1) opposite to that which is fixed the first arm (2.1) is rounded (1.1) eases the introduction of the sleeve member (3.1) on body (1) itself.

3. Surgical instrument, according to claim 1, characterized by the fact that the body (1) consists of a steel rectilinear blade with a rectangular cross-section, having therefore two minor lateral faces and two major lateral faces; the two arms (2,3) rest on a minor lateral face of body (1).
